(19) United States
(12) Patent Application Publication
(10) Pub. No.: US 2025/0191747 A1
(43) Pub. Date: Jun. 12, 2025

(54) SYSTEMS AND DEVICES FOR RECEIVING DATA AND METHODS FOR CONTROL THEREOF

(71) Applicant: ABBOTT DIABETES CARE INC., Alameda, CA (US)

(72) Inventors: Theodore J. Kunich, Pleasanton, CA (US); Xuandon Hua, Mountain View, CA (US); Kevin M. Ow-Wing, Castro Valley, CA (US); Nelson Y. Zhang, Fremont, CA (US)

(73) Assignee: ABBOTT DIABETES CARE INC., Alameda, CA (US)

(21) Appl. No.: 18/842,318

(22) PCT Filed: Apr. 6, 2023
(86) PCT No.: PCT/US2023/017781

§ 371 Date: Aug. 28, 2024

Related U.S. Application Data
(60) Provisional application No. 63/328,037, filed on Apr. 6, 2022.

(51) Int. Cl. G16H 40/60; G01N 33/487

(52) U.S. Cl. CPC G16H 40/60; G01N 33/48707

(57) ABSTRACT

Embodiments described herein include an analyte monitoring device for receiving data in an analyte monitoring system. The analyte monitoring device includes a microprocessor, one or more communications integrated circuits electrically coupled to the microprocessor, wherein the one or more communications integrated circuits are further electrically coupled to at least one respective antenna, an input-output (IO) expander electrically coupled to the microprocessor, and one or more storage memories comprising instructions that, when operable by the microprocessor, cause the microprocessor to receive analyte data from a sensor control device of an analyte sensor in the analyte monitoring system. The IO expander increases an amount of pins of the microprocessor.

### PRIORITY

This application claims the benefit under 35 U.S.C. § 119 (e) of U.S. Provisional Patent Application No. 62/328,037, filed 6 Apr. 2022, which is incorporated herein by reference.

### FIELD

The subject matter described herein relates to systems and devices for receiving data, and methods for control of systems and devices for receiving data, for example, with respect to operation of a handheld device forming part of an in vivo analyte monitoring system.

### BACKGROUND

The frequent monitoring and management of analyte levels, such as glucose, ketones, lactate, oxygen, hemoglobin A1C, or the like, can improve the overall health of people and, in particular, people with diabetes. Patients with diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and can also use this information to determine if and/or when insulin is needed to reduce glucose levels in their bodies, or when additional glucose is needed to raise the level of glucose in their bodies.

Clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device can be worn on the body of an individual who requires analyte monitoring. To increase comfort and convenience for the individual, the sensor control device can have a small form-factor, and can be assembled and applied by the individual with a sensor applicator. The application process includes inserting a sensor, such as an in vivo sensor that senses a user's analyte level in a bodily fluid located in the dermal layer of the human body, using an applicator or insertion mechanism, such that the sensor comes into contact with a bodily fluid. The sensor control device can also be configured to transmit analyte data to one or more data receiving devices, from which the individual, her health care provider (“HCP”), or others can review the data and make therapy decisions. During the life cycle of a sensor, context information can be generated that help improve performance.

In vivo analyte monitoring systems can be broadly classified based on the manner in which data is communicated between the reader device and the sensor control device. One type of in vivo system is a “Continuous Analyte Monitoring” system (or “Continuous Glucose Monitoring” system), where data can be broadcast from the sensor control device to a data receiving device continuously without prompting, e.g., in an automatic fashion according to a broadcast schedule. Another type of in vivo system is a “Flash Analyte Monitoring” system (or “Flash Glucose Monitoring” system or simply “Flash” system), where data can be transferred from the sensor control device in response to a scan or request for data by the data receiving device, such as with a Near Field Communication (NFC) or Radio Frequency Identification (RFID) protocol. A data receiving device can include a variety of hardware components to enable the processing of analyte data received from the sensor control device, and must include telecommunications components to enable the communication with the sensor control device. Data receiving devices can further include additional testing or sending hardware to assist the individual in making therapy decisions.

Accordingly, needs exist for improved analyte monitoring devices and associated data receiving devices greater power efficiency, greater control of performance, and greater hardware and software flexibility to be used in new analyte monitoring applications.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the drawings.

To achieve these and other advantages and in accordance with the purpose of the disclosed subject matter, as embodied and broadly described, the disclosed subject matter includes systems and devices for receiving and processing data in analyte monitoring systems, and methods for control of said systems and devices. Exemplary systems and methods can include a analyte monitoring device for receiving data in an analyte monitoring system, including a microprocessor, one or more communications integrated circuits electrically coupled to the microprocessor, an analog front end electrically coupled to the microprocessor and optionally coupled to a test strip port, an input-output (IO) expander electrically coupled to the microprocessor, and one or more storage memories comprising instructions that, when operable by the microprocessor, cause the microprocessor to receive analyte data from a sensor control device of an analyte sensor in the analyte monitoring system. The one or more communications integrated circuits are further electrically coupled to at least one respective antenna. The analyte monitoring device can optionally be configured, in some examples, to conduct assays to determine a presence or level of an analyte in a sample presented to the test strip port. The IO expander is configured to increase an amount of input and output pins available to the microprocessor. In some embodiments, the analog front end comprises four external op-amps and uses an external VREF. In some embodiments, the IO expander is electrically coupled to a motor for providing haptic feedback, a beeper for providing audible feedback, or a battery monitor integrated circuit.

The microprocessor can include an integrated Universal Serial Bus (USB) module. The instructions can further cause the microprocessor to detect attachment of a USB device through a USB port of the analyte monitoring device and determine a host-type of the USB device, wherein the host-type includes a powered or unpowered USB host. In some embodiments, the microprocessor determines the host-type of the USB device through an attach detect protocol module and battery charging detector module of the analyte monitoring device. In some embodiments, the instructions to cause the microprocessor to detect attachment of the USB device, further cause the microprocessor to measure a ramp time from an attach detect protocol sink voltage to an attach detect protocol probe voltage, compare the ramp time to at least a first threshold and a second threshold, wherein the second threshold is higher than the first threshold, and determine that the ramp time is below the first threshold or above the second threshold. In some embodiments, the analyte monitoring device can include a VBUS protection circuit electrically coupled to the microprocessor. In some embodiments, when the host-type of the USB device is a powered host, the microprocessor prevents use of the analog front end to conduct assays until the USB device is detached.

In some embodiments, the analyte sensor is configured to detect analyte levels in a bodily fluid of a user, wherein a portion of the analyte sensor is configured to be transcutaneously positioned in the user such that when operably positioned, a portion of the analyte sensor is configured to reside above a skin surface of the user, and an in vivo portion of the analyte sensor is configured to reside below the skin surface and in contact with the bodily fluid of the user. In some embodiments, the analyte includes glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, or uric acid

In some embodiments, the analyte monitoring device further includes a display for outputting at least processed analyte data from the sensor control device. The display can include a touchscreen circuit for detecting user input through direct interaction with the display using charge transfer detection between an input node of the touchscreen circuit onto a sampling capacitor of the touchscreen circuit.

In some embodiments, the analyte monitoring device further includes a temperature sensor. The temperature sensor can include a thermistor with a resistance that varies with temperature. The temperature from the sensor can be determined by querying a lookup table mapping the resistance of the thermistor to the detected temperature.

In some embodiments, the microprocessor further includes a hardware-based random number generator. The instructions can further cause the microprocessor to seed a pseudo-random number generator using a random number from the hardware-based random number generator.

According to other aspects of the disclosed subject matter, systems and methods can include a method performed by a microprocessor of an analyte monitoring device in an analyte monitoring system. The method includes, prior to transitioning to a low power mode, enabling write protection of at least one memory location in a flash memory electrically coupled to the microprocessor. The at least one memory location can be associated with a status of one or more status registers in the flash memory. The method includes transitioning, by the microprocessor, to operating in the low power mode. The method includes, after waking from the low power mode, disabling write protection of the at least one memory location, wherein enabling write protection of the at least one memory location prior to transitioning to the low power mode prevents the value stored at the at least one memory location from being unintentionally modified by processes operating while the microprocessor operates in the low power mode. In some embodiments, the status corresponds to a clock security status. In some embodiments, the status corresponds to detection of a sequence execution error.

Other systems, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a high-level diagram depicting example embodiments of an analyte monitoring system for real time analyte (e.g., glucose) measurement, data acquisition and/or processing.

FIG. 2 is a block diagram depicting an example sensor control device.

FIG. 3 is a block diagram depicting an example embodiment of a data receiving device.

FIG. 4 is a schematic diagram of an input/output (IO) expander for use according to embodiments disclosed herein.

FIG. 5 is a schematic diagram of a touchscreen circuit for use according to embodiments disclosed herein.

FIG. 6 is a diagram illustrating touch zone assignment and numbering according to embodiments disclosed herein.

FIG. 7 is a schematic diagram illustrating touchscreen to microprocessor connections according to embodiments disclosed herein.

FIG. 8 is a schematic diagram of a VBUS protection circuit according to embodiments disclosed herein.

FIGS. 9A-9B are schematic diagrams of an analog frontend circuit according to embodiments disclosed herein.

FIG. 10 is a schematic diagram of a temperature sensor according to embodiments disclosed herein.

FIG. 11 illustrates a method 1100 for preventing errors caused by waking a microprocessor.

### DETAILED DESCRIPTION

Reference will now be made in detail to the various exemplary embodiments of the disclosed subject matter, exemplary embodiments of which are illustrated in the accompanying drawings.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

Generally, embodiments of the present disclosure include systems, devices, and methods for the use of analyte sensor insertion applicators for use with in vivo analyte monitoring systems. An applicator can be provided to the user in a sterile package with an electronics housing of the sensor control device contained therein. According to some embodiments, a structure separate from the applicator, such as a container, can also be provided to the user as a sterile package with a sensor module and a sharp module contained therein. The user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid. The embodiments provided herein are improvements to reduce the likelihood that a sensor is improperly inserted or damaged, or elicits an adverse physiological response. Other improvements and advantages are provided as well. The various configurations of these devices are described in detail by way of the embodiments which are only examples.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that can include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “data receiving device,” a “handheld reader device,” “reader device” (or simply a “reader” or “handheld”), a “portable data processing” device or unit, a “data receiver,” a “receiver” device or unit, among other names. Other devices such as personal computers have also been utilized with or incorporated into in vivo and in vitro monitoring systems.

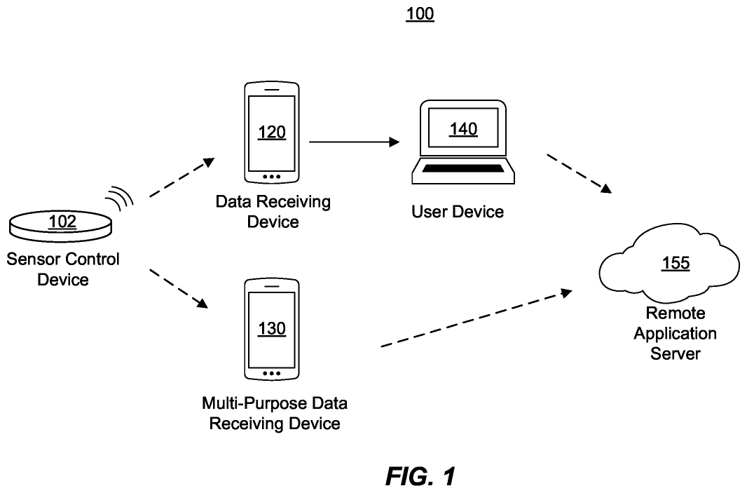

FIG. 1 illustrates an example embodiment of an operating environment of an analyte monitoring system 100 capable of embodying the techniques described herein. As illustrated, the analyte monitoring system 100 can include a system of components designed to provide monitoring of parameters, such as analyte levels, of a human or animal body or can provide for other operations based on the configurations of the various components. As embodied herein, the system can include a low-power sensor control device 102 worn by the user or attached to the body for which information is being collected. As embodied herein, the sensor control device 102 can be a sealed, disposable device with a predetermined active use lifetime (e.g., 1 day, 14 days, 30 days, etc.). Sensor control devices 102 can be applied to the skin of the user body and remain adhered over the duration of the sensor lifetime or can be designed to be selectively removed and remain functional when reapplied. The low-power analyte monitoring system 100 can further include a data reading device 120 or multi-purpose data receiving device 130 configured as described herein to facilitate retrieval and delivery of data, including analyte data, from the sensor control device 102.

As embodied herein, the analyte monitoring system 100 can include a software or firmware library or application provided, for example via a remote application server 155, to a third-party and incorporated into a multi-purpose hardware device 130 such as a mobile phone, tablet, personal computing device, or other similar computing device capable of communicating with the sensor control device 102 over a communication link. Multi-purpose hardware can further include embedded devices, including, but not limited to insulin pumps or insulin pens, having an embedded library configured to communicate with the sensor control device 102. Although the illustrated embodiments of the analyte monitoring system 100 include only one of each of the illustrated devices, this disclosure contemplates the analyte monitoring system 100 incorporate multiples of each components interacting throughout the system. For example and without limitation, as embodied herein, data receiving device 120 and/or multi-purpose data receiving device 130 can include multiples of each. As embodied herein, multiple data receiving devices 130 can communicate directly with sensor control device 102 as described herein. Additionally or alternatively, a data receiving device 130 can communicate with secondary data receiving devices 130 to provide analyte data, or visualization or analysis of the data, for secondary display to the user or other authorized parties.

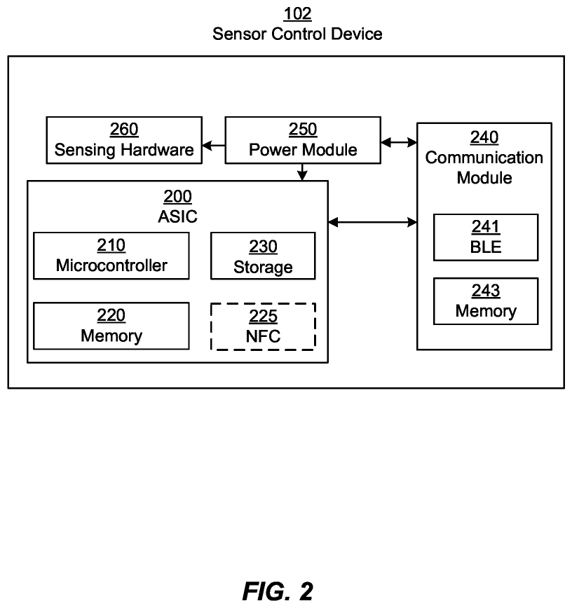

For purpose of illustration and not limitation, FIG. 2 depicts an exemplary embodiment of a sensor control device 102 compatible with the security architecture and communication schemes described herein.

As embodied herein, the sensor control device 102 can include an Application-Specific Integrated Circuit (“ASIC”) 200 communicatively coupled with a communication module 240. The ASIC 200 can include a microcontroller core 210, on-board memory 220, and storage memory 230. The storage memory 230 can store data used in an authentication and encryption security architecture. The storage memory 230 can store programming instructions for sensor control device 102. As embodied herein, certain communication chipsets can be embedded in the ASIC 200 (e.g., an NFC transceiver 225). The ASIC 200 can receive power from a power module 250, such as an on-board battery or from an NFC pulse. The storage memory 230 of the ASIC 200 can be programmed to include information such as an identifier for sensor control device 102 for identification and tracking purposes. The storage memory 230 can also be programmed with configuration or calibration parameters for use by sensor control device 102 and its various components. The storage memory 230 can include rewritable or one-time programming (OTP) memory. The storage memory 230 can be updated using techniques described herein to extend the usefulness of sensor control device 102.

As embodied herein, the communication module 240 of sensor control device 102 can be or include one or more modules to support communications with other devices of an analyte monitoring system 100. As an example only, and not by way of limitation, example communication modules 240 can include a Bluetooth Low-Energy (“BLE”) module 241. As used throughout this disclosure, “BLE” refers to a short-range communication protocol optimized to make pairing of Bluetooth devices simple for end users. The communication module 240 can transmit and receive data and commands via interaction with similarly-capable communication modules of a data receiving device 120 or user device 145. The communication module 240 can include additional or alternative chipsets for use with similar short-range communication schemes, such as a personal area network according to IEEE 802.15 protocols, IEEE 802.11 protocols, infrared communications according to the Infrared Data Association standards (IrDA), etc.

To perform its functionalities, the sensor control device 102 can further include suitable sensing hardware 260 appropriate to its function. As embodied herein, the sensing hardware 260 can include an analyte sensor transcutaneously or subcutaneously positioned in contact with a bodily fluid of a subject. The analyte sensor can generate sensor data containing values corresponding to levels of one or more analytes within the bodily fluid.

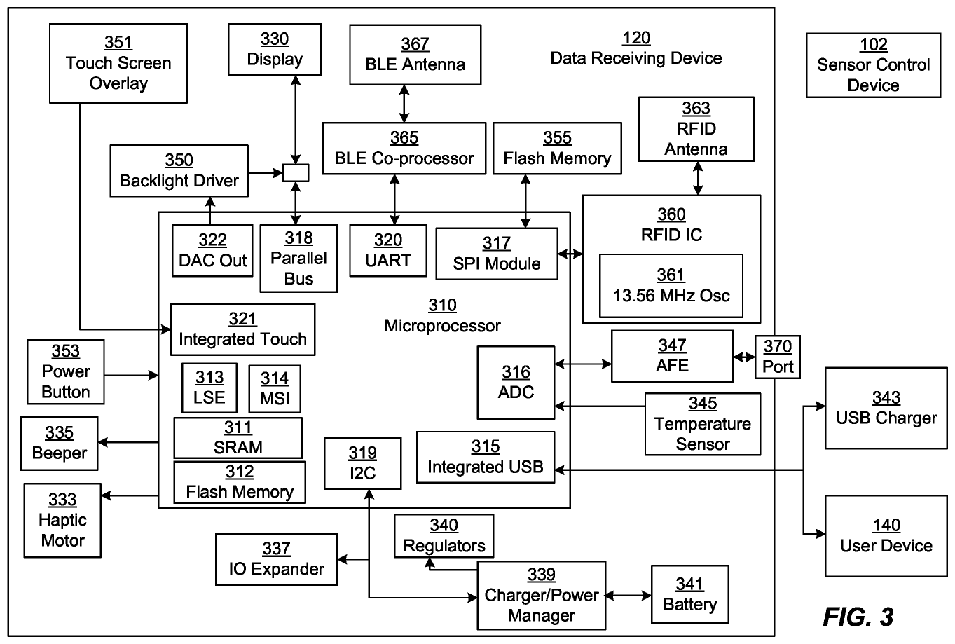

For purpose of illustration and not limitation, reference is made to the exemplary embodiment of a data receiving device 120 for use with the disclosed subject matter as shown in FIG. 3. The data receiving device 120, and the related multi-purpose data receiving device 130, includes components germane to the discussion of the sensor control device 102 and its operations and additional components can be included. In particular embodiments, the data receiving device 120 and multi-purpose data receiving device 130 can be or include components provided by a third party and are not necessarily restricted to include devices made by the same manufacturer as the sensor control device 102.

As illustrated in FIG. 3, the data receiving device 120 includes microprocessor 310 an integrated SRAM 311 and flash memory 312, several oscillators, including a 32.768 kHz low speed external crystal oscillator (LSE) 313 and 48 MHz Multispeed Internal RC oscillator (MSI) 314, and several controllers and busses for receiving and providing input and output. The SRAM 311 can include any suitable size of SRAM for operations of the microprocessor and, as an example only, can include 320 Kbyte accessible memory. Similarly, the internal flash memory 312 can include any suitable size of memory to enable operations of the microprocessor, such as storage of executable code and variables and, as an example only, can include 1 Mbyte accessible memory. In certain embodiments, the microprocessor 310 can be programmed through a serial wired connection consisting of clock, data, reset, and ground. This connection also allows for an interface to a real-time debugger for software development or a flash programmer. The microprocessor 310 includes a variety of reference oscillators which can work in conjunction with external crystals to generate, sources used, as an example, as a reference block for a real-time counter. The real-time counter can be output to various components of the data receiving device 120 as well as for debugging purposes, uses hardware and pin configurations. The microprocessor includes a USB controller 315 and analog-to-digital converter (ADC) 316. An SPI module 317, parallel bus 318, I2C interface 319, and universal asynchronous receiver-transmitter (UART) module 320. As described herein, an integrated touch controller 321 and DAC out 322 further relate to aspects of interactions with a touchscreen display 330.

The data receiving device 120 includes an integrated haptic motor 333 and beeper 335 for providing notifications to a user. The haptic motor 333 provides for haptic feedback from the data receiving device 120 to a user. Haptic feedback can be employed with a counterweight vibration motor mounted to a PCB of the data receiving device 120. The haptic motor 333 is energized by applying a voltage from the battery 341 across motor terminals. The beeper 335 provides audible feedback to the user. The beeper includes an annunciator incorporating a piezo disc. The annunciator can have, as an example, a dual volume and can produces tones at frequencies between 50 and 200 Hz. Maximum sound volume can be generated by causing the annunciator to produce tones at the resonant frequency of the beeper 335 and the cavity in which it resides. Lower sound volume can be less at other frequencies. In particular embodiments, the microprocessor 310 can use one of its available timers to generate a 50% duty cycle pulse-width modulation (PWM) at the desired frequency.

The data receiving device 120 includes a power button 353 enabling the user to manually activate or deactivate the data receiving device 120. The microprocessor 310 can include several power modes, including run, sleep, and stop. When the user is interacting with the data receiving device 120, the microprocessor 310 alternates between run mode to accomplish a particular task and sleep mode once complete. When the data receiving device 120 reader is turned off by the user or due to user inactivity, the microprocessor 310 enters a lower power state, stop, that can be exited via an interrupt. The data receiving device 120 is in this mode when it is off from the user's perspective, only maintaining the real-time clock while waiting for an interrupt to wake up. The interrupt to cause the data receiving device 120 to wake can be issued as a result of the following events: a test strip insert (when a test strip port is available in optional embodiments), a power button 353 press, a USB insertion or a battery low alert. External wakeup input pins can be enabled to detect either rising or falling edges. Any pin can be configured to wake up the microprocessor 310 from all low power modes.

In particular embodiments, certain conditions can cause status bits associated with operation of certain hardware components to be inaccurately reported. As an example, a status bit associated with the low speed oscillator (e.g., the LSE 313) and stored in a reset and clock control register can be incorrectly altered or reported. Specifically, it has been determined that the status bit can be inappropriately set and subsequently cause the low-speed oscillator to never become ready, preventing operation. The conditions causing the error can include waking the microprocessor 310 from certain low power modes or accessing registers to stop the associated timer. In some cases, the hardware associated with the timer register, upon accessing the register (e.g., to stop the timer), can change an otherwise unrelated internal flash memory status register. For example, a bit associated with detecting or reporting a flash memory programming sequence error can be incorrectly set, causing subsequent operations to fail. In some cases, the status bit can be, for example, a clock security status bit, meaning that a corrupted or incorrect value can lead to cascading errors related to security concerns, even if the clock security module was previously disabled.

To prevent this error from occurring, or impacting further operations of the microprocessor, the memory locations associated with the bits that are unintentionally changed (e.g., the battery backup RTC domain) can be temporarily set to a read only state before entering the low power state. This can prevent corruption of the bit, and the rest of the domain, when entering or exiting the low power mode. Additionally or alternatively, given the repeatability of the error, changes to the status bit of an expected nature (e.g., setting the flash memory programming sequence error bit when stopping a particular timer) can be set aside and handled as known errors.

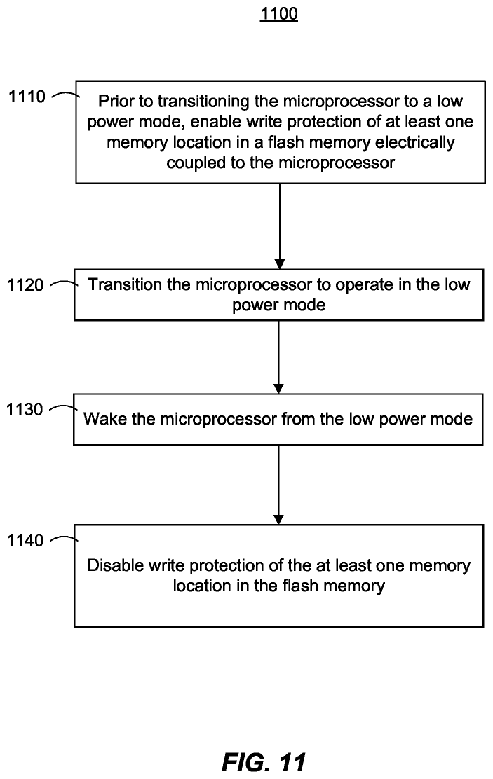

A procedure for preventing or circumventing errors caused by certain known hardware errors is illustrated in FIG. 11. FIG. 11 illustrates a method 1100 for preventing errors caused by waking a microprocessor. At step 1110, prior to transitioning the microprocessor 310 to a low power mode, write protection of at least one memory location in a flash memory 312 electrically coupled to the microprocessor 310 is enabled. The microprocessor 310 can itself enable the write protection, or another memory controller can be included. As an example, the at least one memory location can be associated with a status of one or more status registers in the flash memory 312. The status can be related to security of one or more clocks within or used by the microprocessor 310. The status can be related to detection of execution errors in or associated with the flash memory 312, including, but not limited to a sequence execution error.

At step 1120, the microprocessor 310 is transitioned to operate in the low power mode. At step 1130, the microprocessor 310 is woken up from the low power mode. As described herein, the microprocessor 310 can be woken up through an interrupt issued by or associated with one or more hardware components of the data receiving device 120. Example hardware capable of issuing the interrupt include a power button 353 or touchscreen overlay 351 upon receiving direct user input, a BLE co-processor 365 or RFID IC 360 upon detecting a communication attempt from another device, an ADC 316 upon receiving a signal, for example from an optional AFE 347, an integrated USB module 315, or any other suitable components.

At step 1140 after waking from the low power mode, write protection of the at least one memory location is disabled. As described herein, enabling write protection of the at least one memory location prior to transitioning to the low power mode prevents the value stored at the at least one memory location from being unintentionally modified by processes operating while the microprocessor operates in the low power mode. Disabling write protection upon the microprocessor 310 waking from the low power mode allows for standard operation to continue.

To increase the number of input/output pins available to the microprocessor 310, the data receiving device 120 includes an input/output (IO) expander 337 as described herein. The IO expander 337 is communicatively coupled to the microprocessor 310 via the I2C interface 319.

The inclusion of the IO expander 337 can increase reliance on steady operation of the I2C interface 319, especially when additional peripheral components are added. In some cases, it has been determined that transmit functions on the I2C bus can hang if there is contention over use of the I2C bus (e.g., when several peripherals are attempting to read or write on the I2C bus). This results in an inability of the microprocessor 310 to communication with the peripherals. To mitigate this behavior, the microprocessor 310 can be configured to take preventative or corrective action. As an example, the microprocessor 310 can be configured to detect a failure to communicate via the I2C interface 319. An associated serial data line is checked, and if low, the microprocessor 310 can be configured to change the configuration of the I2C serial clock output to toggle the serial clock a predetermined number of times. Because the “low” state of the serial data line is the incorrect state of an idle I2C bus, toggling the serial clock clears and resets the serial data line. The microprocessor 310 can then check the status of the serial data line for a “high” state, indicating that the I2C bus is now idle.

The I2C interface 319 further couples the microprocessor 310 to a charger/power manager 339 which, in consort with the regulators 340, manages the power supplied to and drawn from the battery 341. The battery 341 is charged when the data receiving device 120 is plugged into an appropriate power source through the integrated USB controller 315. The data receiving device 120 can include USB On-the-Go (OTG) 2.0 Full Speed interface and a suitable connector (e.g., a Micro B Type Connector). The port is used to charge the battery 341 when connected to a USB charger 343, which does not establish a simultaneous data connection, or to interface to a user device 140 to, for example, establish a data connection and download analyte value readings for data analysis and monitoring.

The data receiving device 120 includes an integrated USB compliant with USB battery charger specifications that define limits, detection, control, and reporting mechanisms that permit devices to draw current that exceeds the USB 2.0 specification for charging or powering up from dedicated chargers, hosts, and hubs, and for charging downstream ports. These mechanisms are backward-compatible with USB 2.0 compliant hosts and peripherals. This has led to the creation of USB chargers that expose a USB standard-A receptacle which allows portable devices, such as the data receiving device 120, to use the same USB cable to charge from either a user device 140 or from a USB charger 343.

When connected to a host requiring a data connection, the USB communication is secured by requiring authorization and encryption of payload data. A Meter Abstract Service (MAS) is designed for use during USB communication. The MAS is responsible for establishing communication with the data receiving device 120. The MAS runs in the background on the host device to allow quicker response and load times. The MAS can further begin communicating with the data receiving device 120 and collect data in parallel with auto-launching appropriate applications to enable interpretation of the collected data. The MAS handles authentication with the data receiving device 120 and decrypts data received through the USB connection. The MAS manages plug-ins, operates IPC for client communication, routes events, manages database write access, and provides the client application with notification events such as device connection, detection, and detachment alerts.

The microprocessor 310 includes Battery Charging Detector (BCD) and Attach Detect Protocol (ADP) modules capable of identifying if the data receiving device 120 is connected to a USB Charger 343 (VBUS on) or an unpowered host (VBUS off). Additional hardware provides for data communication. In particular embodiments the actual connection to a USB host can only proceed when these two modules are turned off after use. In some examples, when the data receiving device 120 optionally incorporates an AFE 347 including a test strip port, the data receiving device 120 can be prohibited from doing a test strip assay, e.g., through the AFE 347, anytime it has a galvanic connection to a host computer or USB charger (i.e., via the USB link). If a powered host is connected, a user interface on the display 330 can provide a warning or alert that test strip assays are not permitted.

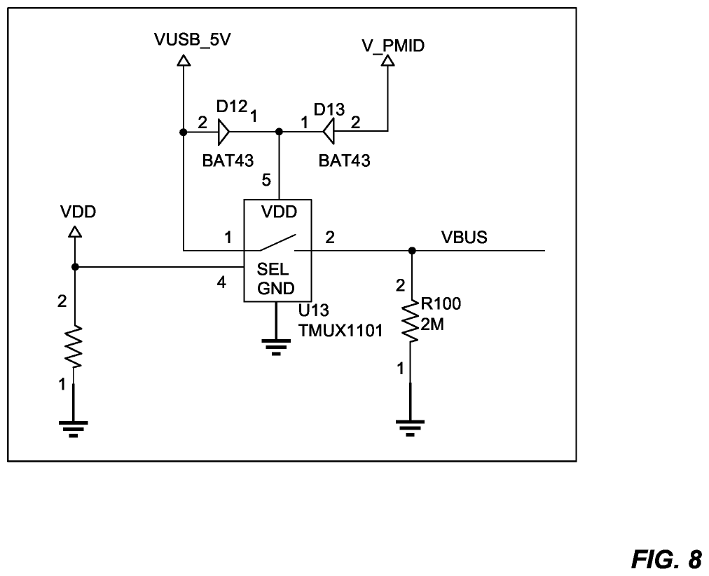

The BCD module of the integrated USB module 315 can detect a standard host port with up to 500 mA maximum draw or a dedicated charging port with up to a 1500 mA maximum draw. The microprocessor 310 requires VBUS to be connected to a specific processor pin. The processor pin can be configured as a GPIO Input, with or without pull-down in order to support ADP. The microprocessor 310 can be protected by ensuring that +5 VDC is not present on PA9 when the microprocessor 310 is unpowered. The circuit illustrated in FIG. 8, which is a schematic diagram of a VBUS protection circuit according to embodiments disclosed herein, can be used to provide this protection. Although VBUS can be used to detect the presence of the USB connection, its absence does not guarantee a non-connection. Therefore, when changes are detected in the 5V state, procedures are initialized to determine whether a powered or unpowered host has been connected.

If VBUS is off, the ADP module can be used to detect an unpowered host according to the following procedure. The procedure can be programmed through software or firmware or other executable code executed by the microprocessor 310. The ADP module measures the VBUS ramp time (RTIM) from the ADP sink voltage to the ADP probe voltage. The ADP driver determines if there is attachment to the USB port of the data receiving device 120 by comparing the RTIM with threshold values. For example, the ADP detection can use two thresholds, which can be set through calibration and during manufacturer. A first, lower, threshold can be associated with detection of an attached host, but an existing voltage on VBUS. A measured RTIM below the first threshold can indicate an unpowered host. A second, higher, threshold can be associated with detection of an attached host and a high external capacitance. A measured RTIM above the second threshold can also indicate an unpowered host. A measured RTIM between the first and second thresholds indicates that no device is connected to the USB port.

From a hardware perspective, the procedure begins with the ADPMEN bits in OTG_GPWRDN register turned on. Then the ADP core is reset by setting the ADPRST bit in the OTG_GADPCTL register. The ENAPRB bit in the OTG_GADPCTL register is then set to start the probe while waiting for the ADPPRBIF bit to be set in the OTG_GADPCTL register to indicate the operation is complete. Next, the RTIM 11 bit field in the OTG_GADPCTL register is read while a timeout check is monitored or while waiting for the ADPTOIF bit to be set. The ADP result is the RTIM in units of 32 kHz, where RTIM is the RC time constant to charge up VBUS to 20%. Therefore, using the ADP detection hardware, the microprocessor 310 can detect the type of device connected to the integrated USB module 315.

The BCD function can be used to detect the battery charger type when a 5V VBUS is available. The BCD function of the integrated USB subsystem 315 can be operated according to the following procedure, which can be programmed through software, firmware, or other executable code executed by the microprocessor 310. First, the BCDEN and DCDEN bits in the OTG_GCCFG register are set and the process waits for the DCDDET bit to come on. A timeout can be employed to detect bad or nonexistent connections. Then, the DCDEN bit is reset, while waiting for a period of time. The PDEN bit is set and the process waits for another period of time. If the PDET bit is set after waiting, then the USB subsystem 315 is connected to a standard downstream port. If PDET is reset after waiting, then the USB subsystem 315 is connected to a dedicated charging port.

Once the type of connection is determined, the USB stack of the microprocessor 310 and integrated USB module 315 provides support for sending and receiving device reports and queuing the reports into commands. Command can include internal commands, text-based commands, or binary commands. Internal commands, which can include firmware updates or writing, can be further supported by the USB stack.

The power subsystem of the data receiving device 120 includes several semi-autonomous functions working in unison. The subsystems include a self-contained battery 341 with a protection circuit module to provide overcharging prevention, over-discharging prevention, and overcurrent prevention and a battery monitor circuit which provides a voltage to an ADC converter input of the microprocessor 310 to measure battery voltage.

The data receiving device 120 can incorporate a power-path management IC to manage battery charging and system power distribution within the reader. Upon connection to a USB port, the data receiving device can be configured conform to the USB standard for current draw during enumeration by appearing as a 100 mA device. Once the upstream device is properly identified as discussed previously, the data receiving device 120 is configured for 400 mA mode, which allows concurrent battery charging and complete device operation from USB power. The upstream device powers the data receiving device 120 while simultaneously and independently charging the battery 341. This reduces the number of charge and discharge cycles on the battery and allows for proper charge termination.

Through the ADC 316, the microprocessor 310 can be communicatively coupled with one or more analog circuits, such as a temperature sensor 345 and an analog frontend (AFE) 347. As described herein, through the AFE 347, the microprocessor 310 of the data receiving device 120 can optionally receive, for example, digital values corresponding to detected levels on test strips.

As described herein, the data receiving device 120 includes an integrated display 330. Output data is presented to the display through a parallel bus 318 with control for the backlight of the display provided through a backlight driver 350. In particular embodiments, the backlight driver 350 out 322 operates as a buffer from the DAC out 322 to reduce the high output impedance. Operating with a buffer in some instances has the effect of lowering the backlight LED current.

In certain embodiments, the display 330 includes an interactive touchscreen. Interactive features are provided through a touch screen overlay 351 that enables addressing of portions of the display 330 which can be mapped to user interface elements.

Although illustrated as a single module 317, the SPI module 317 can encompass multiple distinct modules for operability. The SPI module 317 can interface the microprocessor 310 with an on-board flash memory 355 that is external to the microprocessor 310. The external flash memory 355 can be used to store data such as fonts, bitmaps, application code, and other miscellaneous content. The flash memory 355 or flash memory 312 can further store analyte data received from the sensor control device 102, which the data receiving device communicates with as described herein.

The SPI module 317 additionally interfaces the microprocessor 310 with an RFID integrated circuit 360. The RFID IC 360 can include an oscillator, such as a 13.56 MHz oscillator 361 to enable compliant operations of an RFID antenna 363. The RFID IC 360 can be capable of being clocked up to 10 MHz, but can be limited, for example, to 2 MHz to minimize noise coupling. The RFID IC 360 clock can be configured to 1.5 MHz. As the RFID IC 360 enables communication using, for example, near-field communication protocols, the RFID IC can be configured to generate a signal for the microprocessor 310 associated with an NFC interrupt when incoming communication is detected and/or when servicing of the RFID IC 360 is needed.

The UART module 320 enables the microprocessor 310 to interface with a BLE co-processor 365. The BLE co-processor 365 controls operations of the BLE antenna 367 to enable communication input to and output from the data receiving device 120. Like the RFID IC 360, the BLE co-processor 365 can include an integrated external oscillator to provide timing operations. Both sides, the microprocessor 310 and BLE co-processor 365, support low-power sleep modes. The software used by the microprocessor 310 and BLE co-processor 365 uses hardware pins to wake up the other peer processor to be ready for the incoming command. The wake procedure involves a handshake where the sender asserts the ready signal and wait for the peer processor's ready signal before sending the data. Through control operations of the microprocessor 310, the data receiving device 120 can use the RFID antenna 363 and BLE antenna 367 to communicate with other devices, such as the sensor control device 102.

The data receiving device 120 can be configured to wirelessly couple with the sensor control device 102 and transmit commands to and receive data from the sensor control device 102. As embodied herein, the data receiving device 120 can be configured to operate, with respect to the sensor control device 102 as described herein, as an NFC scanner and a BLE end point. For example, the data receiving device 120 can issue commands (e.g., activation commands for a data broadcast mode of the sensor; pairing commands to identify the data receiving device 120) to the sensor control device 102 using a first module (e.g., the RFID IC 360 and receive data from and transmit data to the sensor control device 102 using a second module (e.g., the BLE co-processor 365 and associated hardware).

As another example, the data receiving device 120 can include, for example, a cellular radio module. The cellular radio module can include one or more radio transceivers for communicating using broadband cellular networks, including, but not limited to third generation (3G), fourth generation (4G), and fifth generation (5G) networks. Additionally, the data receiving device 120 can include a Wi-Fi radio module for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using the cellular radio module or Wi-Fi radio module, the data receiving device 120 can communicate with the remote application server 155 to receive analyte data or provide updates or input received from a user (e.g., through one or more user interfaces).

As embodied herein, the sensor control device 102 can provide data to the data receiving device 120 or multi-purpose data receiving device 130. The data receiving device 120 can transmit the data to the user computing device 145. The user computing device 145 (or the multi-purpose data receiving device 130) can in turn transmit that data to a remote application server 155 for processing and analysis.

As embodied herein, the data receiving device 120 can be configured to operate in coordination with the sensor control device 102 and based on analyte data received from the sensor control device 102. As an example, where the sensor control device 102 is a glucose sensor, the data receiving device 120 can be or include an insulin pump or insulin injection pen. In coordination, the compatible device 130 can adjust an insulin dosage for a user based on glucose values received from the analyte sensor.

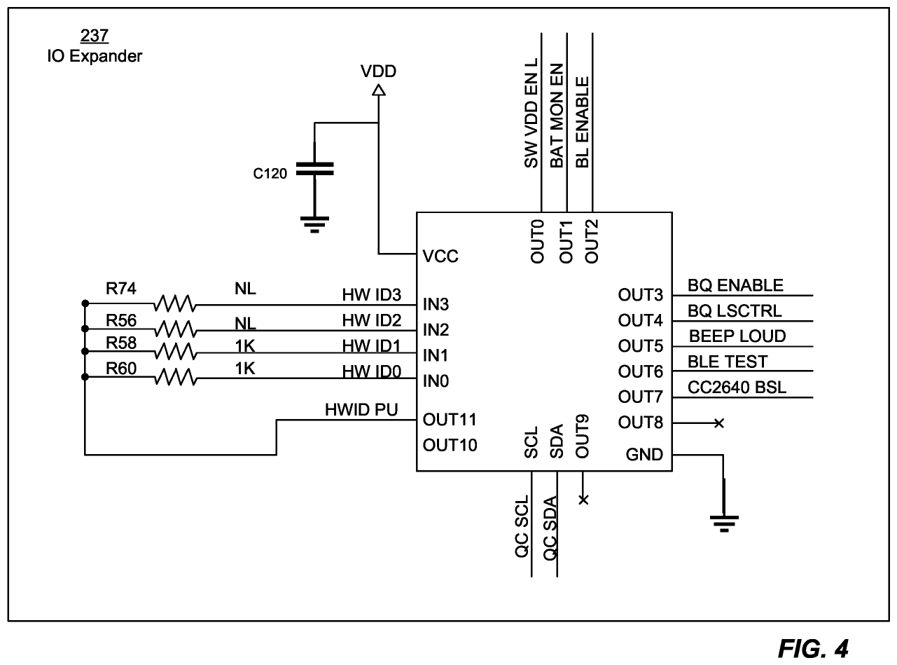

FIG. 4 is a schematic diagram of an input/output (IO) expander 337 for use according to embodiments disclosed herein. The IO expander 337 is used to provide additional pins for input and output access to the microprocessor 310. As an example, the IO expander 337 can be used for interfacing the microprocessor 310 with a battery management IC encompassing the charger/power manager 339 and regulators 340, a switched VDD for the display 330, volume control for the beeper 335 or haptic motor 333 control, or output radios. The microprocessor 310 uses the I2C interface 319 to communicate with the IO expander 337. The outputs (e.g., OUT0-OUT11) can be programmable. The inputs (e.g., IN0-IN3) can have internal resistance pulldowns, for example 100 kOhm pulldowns, within the IO expander.

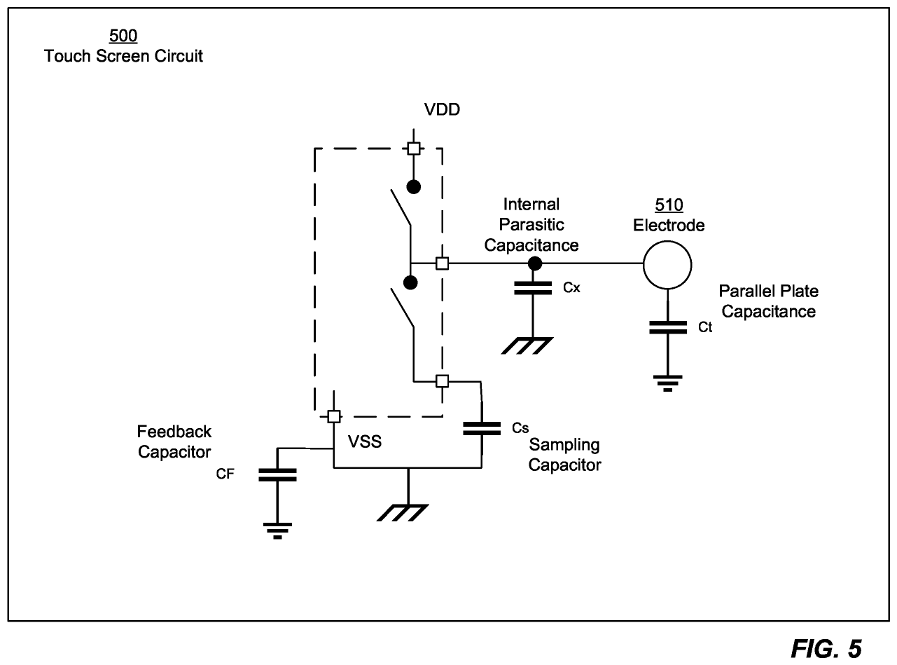

FIG. 5 is a schematic diagram of a touchscreen circuit for use according to embodiments disclosed herein. The touchscreen interface 351 of the display 330 is implemented using an integrated touch sensing controller 321. The touch screen controller module 321 is a hardware touch implementation. The touch screen controller 321 drives the touch electrodes and logic that scans, measures, outputs results, and interrupts the microprocessor 310 based on received input.

The touch sensing feature is based on charge transfer, as shown in FIG. 5. CX includes the internal parasitic capacitance of a general purpose input/output (GPIO) pin of the microprocessor 310 as well as PCB trace capacitance. Both the parasitic capacitance of the GPIO pin and PCB trace capacitance can be referenced to the application ground. Ct is the parallel plate capacitance formed between the electrode 510 and earth ground when a user contacts the electrode, for example using a finger. CF is the feedback capacitor between earth ground and the application ground. Cs is the sampling capacitor used by the touch sensing controller 321. The total capacitance at each touch sensing controller 321 sensor input node is CX+CT//CF.

The touch sensing controller 321 charges the capacitance at the input node to a stable reference voltage (VDD) and then transfers the accumulated charge onto the sampling capacitor (Cs). This process is repeated until the voltage across the sampling capacitor reaches V_IH. The number of transfers needed to reach this threshold is dependent on the amount of capacitance located at the input node. When a user touches the sensor, the capacitance to the earth is increased through the addition of CT, and the amount of time required for the sampling capacitor to reach V_IH is decreased.

When the sampling capacitor reaches V_IH, the touch sensing controller 321 stops the charging process and discharges the sampling capacitor back to 0V. When the number of counts to charge Cs is less than a specified threshold, a detection is reported by the touch sensing controller 321.

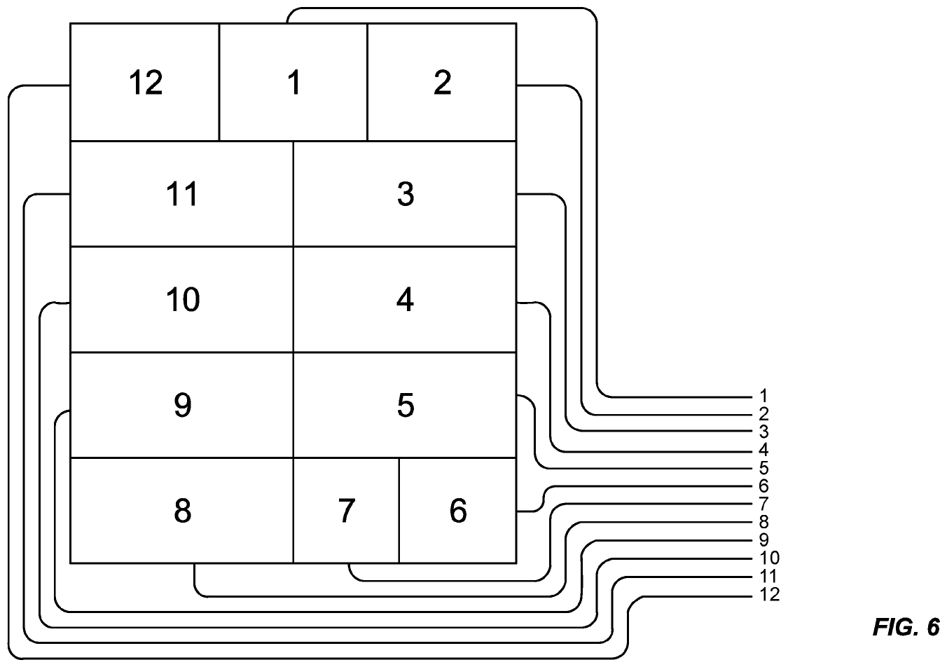

FIG. 6 is a diagram illustrating touch zone assignment and numbering according to embodiments disclosed herein. One sampling capacitor can be used for up to 3 capacitive sensing channels to reduce system components. In particular embodiments, the 3 capacitive sensing channels are connected to sensing zones of approximately the same size and capacitance. The combination of up to 3 sensing channels and one sampling capacitor can be called a touch sensing group.

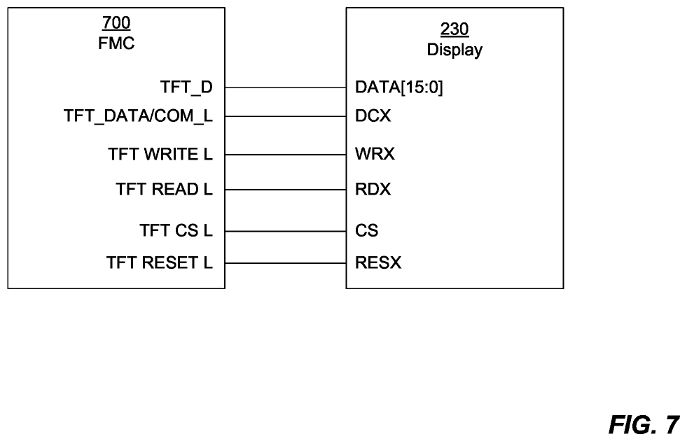

FIG. 7 is a schematic diagram illustrating touchscreen display 330 to microprocessor 310 connections according to embodiments disclosed herein. In particular embodiments, the display 330 is small, relatively low-resolution color TFT display. Other embodiments can include larger or smaller displays, higher or lower resolution, color or non-colored displays, and displays using different display technology. The display controller can be interfaced via several hardware selectable interfaces. In the embodiment shown in FIG. 7, the display uses an 8080 Type 1 16-bit MCU interface. With a 16-bit interface, the data receiving device 120 can support 64K colors (RGB 5-6-5) with a single bus write/read per pixel to the controller.

The microprocessor 310 can include a flexible memory controller (FMC) module 700 that can be configured to interface seamlessly with the display driver. In embodiments in which a display based on TFT technology is used, the backlight of the display 330 can be an integral part of the display module. Unlike LCD interfaces which only need a backlight to view in low light conditions, TFT technology requires a constant light source for the display content to be visible. A boost converter can be configured to act as a voltage controlled current source, pushing a controlled current through a series LED chain within the TFT display module. The boost can draw power directly from the battery 341. DAC control is provided to provide dimmable control of the display, as well as capability of fine tuning brightness if the displays prove to be variable in brightness from one module to another, of one manufacturer to another, etc.

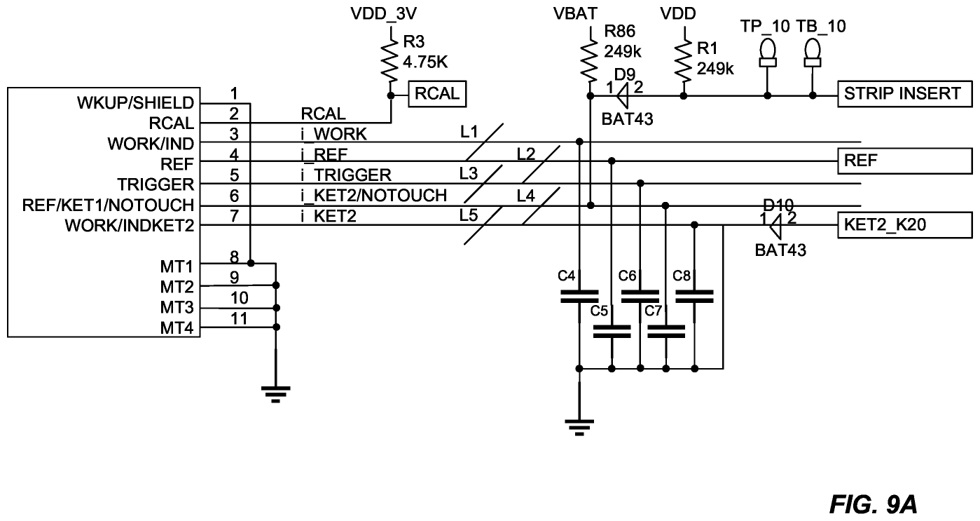

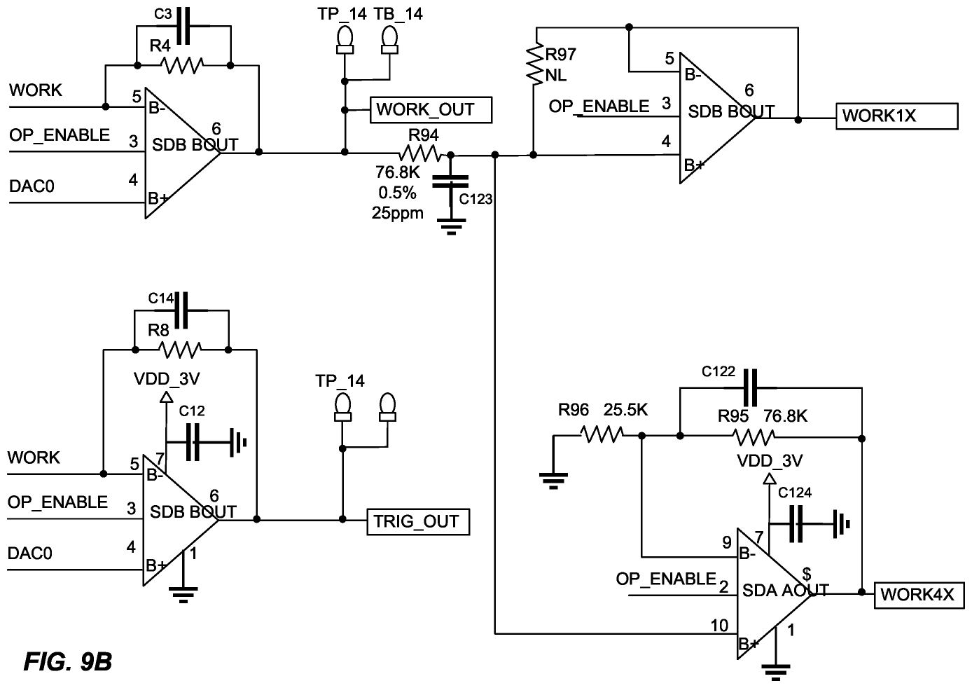

FIGS. 9A-9B are a schematic diagram of an analog frontend circuit according to embodiments disclosed herein. As described, the data receiving device 120 can optionally include hardware and executable code to perform test strip assays. When included and a test strip is inserted into the data receiving device 120, the data receiving device 120 wakes up. A strip insertion and identification process continues. First, the strip insertion causes an interrupt as a signal is grounded mechanically by strip port 370. This grounds the pull-up resistor to VBAT. Next, the firmware tests for the type of test strip. As an example, a ketones test strip can be identified by detecting a conductive bar printed to short strip specific port pins together. The test strip AFE 347 detects the short and set an appropriate pin to issue an interrupt to the ADC 316. If a test strip is detected, but cannot be identified as a ketones test strip, then the firmware can assume that the test strip is a glucose test strip and proceed accordingly. In other embodiments, the glucose test strip can be affirmatively determined and a ketones test strip can be default assumed. Although ketone and glucose test strips are discussed, this is merely by way of illustrative example and additional types of test strips can be encountered.

Referring to FIGS. 9A-9B, external op-amps U1A and U1B are trans-impedance amplifiers for TRIGGER and WORK, external op-amp U12B is a fixed unity gain buffer for the R-C filtered work 1× channel, WORK FILT, external op-amp U12A is a fixed 4× gain amplifier for the work 4× channel. DACO is a 12-bit DAC for work and trigger trans-impedance amplifier biasing. These circuits support a variety of AFE channels and current ranges. An assay phase is identified for each channel, which can include, but are not limited to work peak, work 1×, work 4×, and trigger. In particular embodiments, a similar circuit as shown in FIGS. 9A-9B can be implemented using two available op-amps inside the microprocessor 310 for the work 1× buffer and the work 4× gain stage.

In particular embodiments, a 3V ADC reference is used to reduce the use of a differential amp. This configuration can allow for a 4× gain with the largest input bias, which leaves sufficient headroom for the work 4× signal. A diff-amp is not used to subtract the strip bias voltages prior to applying the 4× gain to get the work 4× signal. This avoids the need for a bipolar ADC mode to handle negative readings when the signal is near zero. This configuration also reduces the need to add a bias to the differential signal to keep it positive at the A/D input. However, this configuration requires the use of a 1.2V band gap reference, VREFINT, inside the microprocessor 310 to correct ADC data and DAC settings due to changes in VREF+, which can drift due to a slowly decreasing or discharging battery voltage input to the LDO. To use a 16-bit resolution, hardware oversampling and software averaging is used. In general, a greater ADC resolution is useful as a large component of conversion error is often in internal ADC noise in counts. So, the better the resolution, the lower the chance that noise can present issues in terms of the current it represents. The ADC used by the microprocessor 310 can be upscaled to a higher resolution through hardware oversampling and software averaging. A hardware oversampling setting of 16× or more gives 16-bit results via summation of successive ADC samples and reduction to 16-bits.

There are four strip AFE signals, which are output of four different op-amps in the circuit illustrated in FIG. 8. The op-amp outputs then go into four different ADC inputs. Current range, resolution, bandwidth, and time constants are correlated and information directly applicable to software control and range switching of the AFE is extracted. Tolerances can be built into the nominal design resolution and range specifications since op-amp resistor and ADC reference voltage tolerances result in minor deviations from max total gain and range tolerance from nominal. Since the work 4× gain stage follows a 300 Hz LPF, a lag-lead filter topology is used to place the filter well beyond the target frequency. The lag-lead filter, which means that the frequency response is typically a 4× gain low-pass filter with a cut-off at a pole frequency, then approaches unity gain at frequencies beyond the zero frequency.

In particular embodiments, default and assay ADC samples can be performed using a maximum hardware oversampling (of 256×) to reduce the added overhead of performed software averaging for default samples. This approach simplifies implementation details and standardize implementation requirements across sample types. However, in some cases, unintentional errors can prevent this approach. As an example, in some cases hardware bugs can spoil the first ADC sample processed under this approach. A work-around can be to discard the first sample. However, because the assay timing is given in 1 millisecond resolution, a 256× oversampling creates a sample that is too long to discard. Therefore, 8× resolution hardware oversampling can be used which reduces the time error of a first discarded sample to less than 1 millisecond. Software averaging can be used to obtain a 256× average of default hardware samples.

In particular embodiments, rather than correct each ADC sample or sample average during strip assays, the strip signal channel slopes (e.g., for work peak, work 1×, work 4×, and trigger) can be corrected and stored at the beginning of easy assay. These corrected slopes can be applied to the raw ADC strip signal readings to get the assay current used for determining the analyte values for an assay. A similar scheme can be employed for DAC setting corrections.

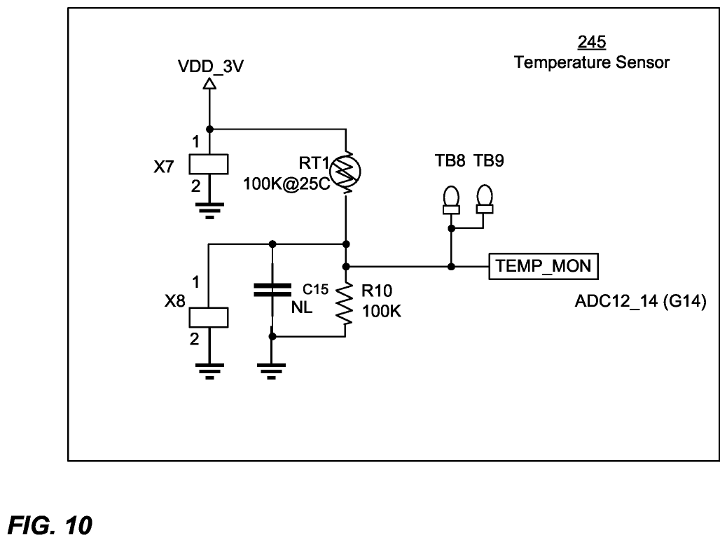

FIG. 10 is a schematic diagram of a temperature sensor 345 according to embodiments disclosed herein. The data receiving device 120 can measure a temperature within a specified tolerance accuracy (e.g., of +1° C.), over the operating temperature range. As illustrated in FIG. 10, the temperature sensor includes an external 100KΩ (at 25° C.) thermistor, RT1, the resistance of which varies with temperature. The thermistor is used for temperature sensing. A mapping of temperature vs RT1 measurement can be provided by the manufacturer. A 100KΩ resistor is further placed in series with RT1 to generate a voltage, TEMP_MON, the digital conversion of which is used in a software look-up table (LUT) to determine the temperature. Note that the thermistor is driven by a 3V VDD, which causes a small self-heating effect at the thermistor. However, this effect is on the order of about 0.02° C., which is negligible compared to the +1° temperature measurement accuracy requirement. Detected temperatures from the temperature sensor can be used to calibrate or otherwise modify the analyte values interpreted by the microprocessor 310.

The external work and trigger op-amps are enabled via GPIO control of OP_ENABLE and OP1_ENABLE from the microprocessor 310. OP_ENABLE enables or disables the three work channel external op-amps—WORK_OUT, WORK1× and WORK4×—together. OP_ENABLE1 enables or disables the Trigger external op-amp. Open-drain 3V+ tolerant microprocessor 310 output pins are used to interface the 3V op-amp enable inputs to the 1.8V microprocessor 310 I/O. 249K external pull-ups are used for each op-amp enable. This choice of pull-up resistor value results in open-drain microprocessor 310 high and low outputs with a value close to the op-amp supply rails, providing good logic threshold margin.

A strip REF pin is assigned to two microprocessor 310 output pins connected together to reduce low voltage output saturation. The output pins can change states together simultaneously under software control. The strip REF is used to suspend the chemical reaction during the assay when the strip REF is at high-impedance. The strip REF is used to initiate the chemical reaction during the assay when the strip REF is at low-impedance. The low voltage on this pin saturates at 1.0 mV nominal at the highest specified Work current level of 120 uA.

ADC self-calibration is used to adjust the ADC offset. ADC offset calibration is performed every time the data receiving device 120 wakes up, allowing for the ADC self-calibration to be performed prior to run-time assay offset measurement. In particular embodiments, ADC self-calibration can also be performed prior to AFE channel slope calibration or prior to AFE ADC serial commands.

The storage memory 230 of the sensor control device 102 can include the software blocks related to communication protocols of the communication module. For example, the storage memory 230 can include a BLE services software block with functions to provide interfaces to make the BLE module 241 available to the computing hardware of the sensor control device 102. These software functions can include a BLE logical interface and interface parser. BLE services offered by the communication module 240 can include the generic access profile service, the generic attribute service, generic access service, device information service, data transmission services, and security services. The data transmission service can be a primary service used for transmitting data such as sensor control data, sensor status data, analyte measurement data (historical and current), and event log data. The sensor status data can include error data, current time active, and software state. The analyte measurement data can include information such as current and historical raw measurement values, current and historical values after processing using an appropriate algorithm or model, projections and trends of measurement levels, comparisons of other values to patient-specific averages, calls to action as determined by the algorithms or models and other similar types of data.

According to aspects of the disclosed subject matter, and as embodied herein, a sensor control device 102 can be configured to communicate with multiple devices concurrently by adapting the features of a communication protocol or medium supported by the hardware and radios of the sensor control device 102. As an example, the BLE module 241 of the communication module 240 can be provided with software or firmware to enable multiple concurrent connections between the sensor control device 102 as a central device and the other devices as peripheral devices, or as a peripheral device where another device is a central device.

Connections, and ensuing communication sessions, between two devices using a communication protocol such as BLE can be characterized by a similar physical channel operated between the two devices (e.g., a sensor control device 102 and data receiving device 120). The physical channel can include a single channel or a series of channels, including for example and without limitation using an agreed upon series of channels determined by a common clock and channel- or frequency-hopping sequence. Communication sessions can use a similar amount of the available communication spectrum, and multiple such communication sessions can exist in proximity. In certain embodiment, each collection of devices in a communication session uses a different physical channel or series of channels, to manage interference of devices in the same proximity.

For purpose of illustration and not limitation, reference is made to an exemplary embodiment of a procedure for a sensor-receiver connection for use with the disclosed subject matter. First, the sensor control device 102 repeatedly advertises its connection information to its environment in a search for a data receiving device 120. The sensor control device 102 can repeat advertising on a regular basis until a connection established. The data receiving device 120 detects the advertising packet and scans and filters for the sensor control device 102 to connect to through the data provided in the advertising packet. Next, data receiving device 120 sends a scan request command and the sensor control device 102 responds with a scan response packet providing additional details. Then, the data receiving device 120 sends a connection request using the Bluetooth device address associated with the data receiving device 120. The data receiving device 120 can also continuously request to establish a connection to a sensor control device 102 with a specific Bluetooth device address. Then, the devices establish an initial connection allowing them to begin to exchange data. The devices begin a process to initialize data exchange services and perform a mutual authentication procedure.

During a first connection between the sensor control device 102 and data receiving device 120, the data receiving device 120 can initialize a service, characteristic, and attribute discovery procedure. The data receiving device 120 can evaluate these features of the sensor control device 102 and store them for use during subsequent connections. Next, the devices enable a notification for a customized security service used for mutual authentication of the sensor control device 102 and data receiving device 120. The mutual authentication procedure can be automated and require no user interaction. Following the successful completion of the mutual authentication procedure, the sensor control device 102 sends a connection parameter update to request the data receiving device 120 to use connection parameter settings preferred by the sensor control device 102 and configured to maximum longevity.

The data receiving device 120 can then perform sensor control procedures to backfill historical data, current data, event log, and factory data. As an example, for each type of data, the data receiving device 120 can send a request to initiate a backfill process. The request can specify a range of records defined based on, for example, the measurement value, timestamp, or similar, as appropriate. The sensor control device 102 responds with requested data until all previously unsent data in the memory of the sensor control device 102 is delivered to the data receiving device 120. The sensor control device 102 can respond to a backfill request from the data receiving device 120 that all data has already been sent. Once backfill is completed, the data receiving device 120 can notify sensor control device 102 that it is ready to receive regular measurement readings. The sensor control device 102 can send readings across multiple notifications result on a repeating basis. As embodied herein, the multiple notifications can be redundant notifications to ensure that data is transmitted correctly. Alternatively, multiple notifications can make up a single payload.

For purpose of illustration and not limitation, reference is made to an exemplary embodiment of a procedure to send a shutdown command to the sensor control device 102. The shutdown operation is executed if the sensor control device 102 is in, for example, an error state, insertion failed state, or sensor expired state. If the sensor control device 102 is not in those states, the sensor control device 102 can log the command and execute the shutdown when sensor control device 102 transitions into the error state or sensor expired state. The data receiving device 120 sends a properly formatted shutdown command to the sensor control device 102. If the sensor control device 102 is actively processing another command, the sensor control device 102 responds with a standard error response indicating that the sensor control device 102 is busy. Otherwise, the sensor control device 102 sends a response as the command is received. Additionally, the sensor control device 102 sends a success notification through the sensor control characteristic to acknowledge the sensor control device 102 has received the command. The sensor control device 102 registers the shutdown command. At the next appropriate opportunity (e.g., depending on the current sensor state, as described herein), the sensor control device 102 shuts down.

The microprocessor 310 can further provide a hardware-based random number generator. The random number generator can be used to facilitate certain authentication or encryption operations by providing a nonce value, check value, or other initialization value used when two devices are confirming a shared secret or access to a private key by a peer device. Authentication and encryption operations can be used, for example, to support NFC, BLE, or USB security. A hardware-based random number generator provided by the microprocessor 310 can improve on configurations in which only software-based random number generator is available. As software-based approaches can be deterministic, they are dependent on the seed values used and therefore are considered pseudo-random number generators. Additionally, in certain configurations, a hardware-based random number generator can be provided by a peer processor of the data receiving device 120 such as the BLE co-processor 365. In such configurations, when a random number generator is required, the microprocessor 310 requests a random number from the peer processor. The microprocessor 310 can use the random number received from the peer processor or can use the random number as a seed for a software-based approach. However, these approaches can have undesirable side effects. As an example, requesting a hardware-based random number from a peer processor, such as the BLE co-processor 365 slows down processes reliant on the random number, by invoking a form of input and output (from the perspective of the processors). Additionally, involving a new hardware component removes the control of the random number generation from the perspective of the microprocessor 310, which can expose unintentional errors or security risks. Moreover, requiring an additional hardware component to generate a random number consumes additional battery power when compared to using a hardware-based approach as provided by the microprocessor 310. Therefore, a hardware-based random number generator incorporated in the microprocessor itself 120 can increase availability of random numbers (encouraging their use), reduce the time, energy and input-output related costs of using hardware-based random numbers generated by a co-processor, and minimize exposure to intentional abuse or unintentional errors.

Multi-purpose data receiving device 130 can be a mobile communication device such as, for example, a Wi-Fi or internet enabled smartphone, tablet, or personal digital assistant (PDA). Examples of smartphones can include, but are not limited to, those phones based on a WINDOWS operating system, ANDROID operating system, IPHONE operating system, PALM, WEBOS, BLACKBERRY operating system, or SYMBIAN operating system, with data network connectivity functionality for data communication over an internet connection and/or a local area network (LAN).

Multi-purpose data receiving device 130 can also be configured as a mobile smart wearable electronics assembly, such as an optical assembly that is worn over or adjacent to the user's eye (e.g., a smart glass or smart glasses, such as GOOGLE GLASSES). This optical assembly can have a transparent display that displays information about the user's analyte level (as described herein) to the user while at the same time allowing the user to see through the display such that the user's overall vision is minimally obstructed. The optical assembly can be capable of wireless communications similar to a smartphone. Other examples of wearable electronics include devices that are worn around or in the proximity of the user's wrist (e.g., a smart watch, etc.), neck (e.g., a necklace, etc.), head (e.g., a headband, hat, etc.), chest, or the like.

The multi-purpose data receiving device 130 can be configured with a software application package (e.g., an “app”) downloadable from a remote application server 155 or application store. The app can enable communication with the sensor control device 102 using one or more protocols compatible with the communication hardware of the sensor control device (e.g., the BLE module 241 or NFC module 225). The app can further enable the multi-purpose data receiving device 130 to process data received from the sensor control device 102. For example, the app can enable the multi-purpose data receiving device 130 to decrypt and interpret analyte data for display to a user.

1. An analyte monitoring device for receiving data in an analyte monitoring system, comprising:
a microprocessor;
one or more communications integrated circuits electrically coupled to the microprocessor, wherein the one or more communications integrated circuits are further electrically coupled to at least one respective antenna;
an analog front end electrically coupled to the microprocessor and coupled to a test strip port, wherein the analyte monitoring device is configured to conduct assays to determine a presence or level of an analyte in a sample presented to the test strip port;
an input-output (IO) expander electrically coupled to the microprocessor, wherein the IO expander is configured to increase an amount of input and output pins available to the microprocessor; and
one or more storage memories comprising instructions that, when operable by the microprocessor, cause the microprocessor to receive analyte data from a sensor control device of an analyte sensor in the analyte monitoring system.

2. The analyte monitoring device of claim 1, wherein the microprocessor comprises an integrated Universal Serial Bus (USB) module, and wherein the instructions further cause the microprocessor to:
detect attachment of a USB device through a USB port of the analyte monitoring device; and
determine a host-type of the USB device, wherein the host-type includes a powered or unpowered USB host.

3. The analyte monitoring device of claim 2, wherein the microprocessor determines the host-type of the USB device through an attach detect protocol module and battery charging detector module of the analyte monitoring device.

4. The analyte monitoring device of claim 3, wherein the instructions to cause the microprocessor to detect attachment of the USB device, further cause the microprocessor to:
measure a ramp time from an attach detect protocol sink voltage to an attach detect protocol probe voltage;
compare the ramp time to at least a first threshold and a second threshold, wherein the second threshold is higher than the first threshold; and
determine that the ramp time is below the first threshold or above the second threshold.

5. The analyte monitoring device of claim 2, further comprising a VBUS protection circuit electrically coupled to the microprocessor.

6. The analyte monitoring device of claim 2, wherein, when the host-type of the USB device is a powered host, the microprocessor prevents use of the analog front end to conduct assays until the USB device is detached.

7. The analyte monitoring device of claim 1, wherein the analyte sensor is configured to detect analyte levels in a bodily fluid of a user, wherein a portion of the analyte sensor is configured to be transcutaneously positioned in the user such that when operably positioned, a portion of the analyte sensor is configured to reside above a skin surface of the user, and an in vivo portion of the analyte sensor is configured to reside below the skin surface and in contact with the bodily fluid of the user.

8. The analyte monitoring device of claim 1, further comprising a display for outputting at least processed analyte data from the sensor control device, wherein the display comprises a touchscreen circuit for detecting user input through direct interaction with the display using charge transfer detection between an input node of the touchscreen circuit onto a sampling capacitor of the touchscreen circuit.

9. The analyte monitoring device of claim 1, further comprising a temperature sensor comprising a thermistor with a resistance that varies with temperature, wherein the temperature from the sensor is determined by querying a lookup table mapping the resistance of the thermistor to the detected temperature.

10. The analyte monitoring device of claim 1, wherein the microprocessor further comprises a hardware-based random number generator.

11. The analyte monitoring device of claim 10, wherein the instructions further cause the microprocessor to seed a pseudo-random number generator using a random number from the hardware-based random number generator.

12. The analyte monitoring device of claim 1, wherein the analyte comprises glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, or uric acid.

13. The analyte monitoring device of claim 1, wherein the analog front end comprises four external op-amps and uses an external VREF.

14. The analyte monitoring device of claim 1, the microprocessor is electrically coupled to a motor for providing haptic feedback.

15. The analyte monitoring device of claim 1, wherein the IO expander is electrically coupled to a beeper for providing audible feedback.

16. The analyte monitoring device of claim 1, wherein the IO expander is electrically coupled to a battery monitor integrated circuit of the analyte monitoring device.

17. A method comprising, by a microprocessor of an analyte monitoring device in an analyte monitoring system:
prior to transitioning to a low power mode, enabling write protection of at least one memory location in a flash memory electrically coupled to the microprocessor, wherein the at least one memory location is associated with a status of one or more status registers in the flash memory; transitioning, by the microprocessor, to operating in the low power mode; and
after waking from the low power mode, disabling write protection of the at least one memory location, wherein enabling write protection of the at least one memory location prior to transitioning to the low power mode prevents the value stored at the at least one memory location from being unintentionally modified by processes operating while the microprocessor operates in the low power mode.

18. The method of claim 17, wherein the status corresponds to a clock security status.

19. An analyte monitoring device for receiving data in an analyte monitoring system, comprising:
a microprocessor;
one or more communications integrated circuits electrically coupled to the microprocessor, wherein the one or more communications integrated circuits are further electrically coupled to at least one respective antenna;
an input-output (IO) expander electrically coupled to the microprocessor, wherein the IO expander is configured to increase an amount of input and output pins available to the microprocessor; and
one or more storage memories comprising instructions that, when operable by the microprocessor, cause the microprocessor to receive analyte data from a sensor control device of an analyte sensor in the analyte monitoring system.

20. The analyte monitoring device of claim 19, wherein the microprocessor comprises an integrated Universal Serial Bus (USB) module, and wherein the instructions further cause the microprocessor to:
detect attachment of a USB device through a USB port of the analyte monitoring device; and
determine a host-type of the USB device, wherein the host-type includes a powered or unpowered USB host.

21. The analyte monitoring device of claim 20, wherein the microprocessor determines the host-type of the USB device through an attach detect protocol module and battery charging detector module of the analyte monitoring device.

22. The analyte monitoring device of claim 21, wherein the instructions to cause the microprocessor to detect attachment of the USB device, further cause the microprocessor to:
measure a ramp time from an attach detect protocol sink voltage to an attach detect protocol probe voltage;
compare the ramp time to at least a first threshold and a second threshold, wherein the second threshold is higher than the first threshold; and
determine that the ramp time is below the first threshold or above the second threshold.

23. The analyte monitoring device of claim 20, wherein, when the host-type of the USB device is a powered host, the microprocessor prevents use of the analog front end to conduct assays until the USB device is detached.

24. The analyte monitoring device of claim 19, further comprising a VBUS protection circuit electrically coupled to the microprocessor.

25. The analyte monitoring device of claim 19, wherein the analyte sensor is configured to detect analyte levels in a bodily fluid of a user, wherein a portion of the analyte sensor is configured to be transcutaneously positioned in the user such that when operably positioned, a portion of the analyte sensor is configured to reside above a skin surface of the user, and an in vivo portion of the analyte sensor is configured to reside below the skin surface and in contact with the bodily fluid of the user.

26. The analyte monitoring device of claim 19, further comprising a display for outputting at least processed analyte data from the sensor control device.

27. The analyte monitoring device of claim 26, wherein the display comprises a touchscreen circuit for detecting user input through direct interaction with the display using charge transfer detection between an input node of the touchscreen circuit onto a sampling capacitor of the touchscreen circuit.

28. The analyte monitoring device of claim 19, further comprising a temperature sensor comprising a thermistor with a resistance that varies with temperature, wherein the temperature from the sensor is determined by querying a lookup table mapping the resistance of the thermistor to the detected temperature.

29. The analyte monitoring device of claim 19, wherein the microprocessor further comprises a hardware-based random number generator.

30. The analyte monitoring device of claim 29, wherein the instructions further cause the microprocessor to seed a pseudo-random number generator using a random number from the hardware-based random number generator.

31. The analyte monitoring device of claim 19, wherein the analyte comprises glucose, ketones, lactate, oxygen, hemoglobin A1 C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, or uric acid.

32. The analyte monitoring device of any of claims 19-31 claim 19,
wherein the microprocessor is electrically coupled to a motor for providing haptic feedback.

33. The analyte monitoring device of claim 19, wherein the IO expander is electrically coupled to a beeper for providing audible feedback.

34. The analyte monitoring device of claim 19, wherein the IO expander is electrically coupled to a battery monitor integrated circuit of the analyte monitoring device.

35. The analyte monitoring device of claim 19, further comprising an analog front end electrically coupled to the microprocessor and coupled to a test strip port, wherein the analyte monitoring device is configured to conduct assays to determine a presence or level of an analyte in a sample presented to the test strip port.

36. The analyte monitoring device of claim 35, wherein the analog front end comprises four external op-amps and uses an external VREF.
